(19) United States
(12) Patent Application Publication
(10) Pub. No.: US 2025/0090609 A1
(43) Pub. Date: Mar. 20, 2025

(54) COMPOSITIONS AND METHODS FOR PROMOTING DEVELOPMENT OF INFANT

(71) Applicant: Bobbie Baby, Inc., San Francisco, CA (US)

(72) Inventors: Christina BERBERICH, Santa Cruz, CA (US); Pedro PRIETO, Columbus, OH (US); Danielle ADDISON, Denver, CO (US)

(21) Appl. No.: 18/818,847

(22) Filed: Aug. 29, 2024

Related U.S. Application Data
(60) Provisional application No. 63/538,619, filed on Sep. 15, 2023.

(51) Int. Cl. A61K 36/02; A61K 31/145; A61K 31/205; A61K 31/375; A61K 31/519; A61K 31/593; A61K 31/714; A61K 33/26; A61K 36/06; A61K 45/06; A61P 3/02

(52) U.S. Cl. CPC A61K 36/02; A61K 31/145; A61K 31/205; A61K 31/375; A61K 31/519; A61K 31/593; A61K 31/714; A61K 33/26; A61K 36/06; A61K 45/06; A61P 3/02

(57) ABSTRACT

Aspects of the present disclosure provide, inter alia, a novel nutritional formula for infants and toddlers. Also provided are methods for promoting development of infants and toddlers, such as development of the brain, eye and/or immune system, including in preterm infants.

### CROSS REFERENCE TO RELATED APPLICATIONS

The present application claims benefit of U.S. Provisional Patent Application Ser. No. 63/538,619, filed on Sep. 15, 2023. The entire content of the aforementioned application is incorporated by reference as if recited in full herein.

### FIELD OF DISCLOSURE

Aspects of the present disclosure provides, inter alia, a novel nutritional formula for infants and toddlers. Also provided are methods for promoting development of infants, such as brain and/or eye development of infants, and in particular preterm infants.

Folate is an essential vitamin in humans. Substantial evidence supports the role of folic acid's effectiveness in the prevention of neural tube defects (NTD's) (De Wals et al. 2008; Berry et al. 1999). NTD's are congenital malformations of structures of the central nervous system that occur due to the failure of neural tube closure after conception between 21 and 28 days (Blencowe et al. 2010). Elevated blood homocysteine may increase the risk of stroke and coronary heart diseases and normal folate metabolism has been shown to reduce homocysteine levels (Cui et al. 2010). Evidence from studies suggest low maternal blood folate during early pregnancy may be linked to behavioral disorders in childhood and folate may also be a key factor involved in cognitive function such as Alzheimer's disease (Schlotz et al. 2009; Smith, 2008). Folate status has been linked to the development of other chronic diseases as well as in reproductive health (Naderi, 2018).

Docosahexaenoic acid (DHA), a long chain polyunsaturated fatty acid (LCPUFA), is highly enriched in neural membranes, accounting for about 30% to 40% of gray matter of the cerebral cortex and photoreceptors in the retina and it is the predominant fatty acid of the retina. Specific functional roles in addition to normal cell membrane function are attributed to the high concentrations of DHA in these tissues (Tai et al. 2013; Bradbury, 2011; Horrocks et al. 1999). Evidence shows that retinal lipid composition influences the ability of visual pigment rhodopsin to generate visual nerve impulses. Of note, fatty acids in the retinal membrane other than DHA are typically not efficient substitutes for proper visual signal transduction, which underscores the importance of DHA (Innis, 1991). DHA has been shown to be actively involved in neuronal development and plasticity, receptor-mediated signaling, changes in membrane fluidity, the formation of secondary messengers, and enhancement of the production of anti-inflammatory lipid mediators (Bradbury, 2011; Horrocks et al. 1999). DHA also serves as a substrate for the activities of the enzymes lipoxygenase and cyclooxygenase, thus preventing the formation of pro-inflammatory arachidonic acid products (Bradbury, 2011; Horrocks et al. 1999). In addition, DHA has been found to act as an agonist or an antagonist of several receptors such as plasma membrane bound Toll-like receptors (TLR), the G-coupled protein receptor (GPR) 120, the nuclear receptor peroxisome proliferator activated receptor gamma (PPARy), and the dopamine and serotonin receptors (Horrocks et al. 1999).

Arachidonic acid (ARA), a LCPUFA that is also present in the brain, is distributed more widely than DHA in the human body. ARA is the primary unsaturated fatty acid in the heart, muscle, vascular endothelium, T-lymphocytes, adrenal gland, kidneys, liver, and the placenta (Hadley et al. 2016). ARA is crucial for brain development by influencing cell division and signaling. The rapid accumulation of ARA in the brain during development starts from the beginning of the third trimester of gestation and lasts up to about 2 years of age. Brain growth rate is directly associated with myelination and diets low in LCPUFA deter the development of the myelin lipids necessary in brain development (Hadley et al. 2016; Tounian et al. 2021). Notably, ARA is the precursor of another LCPUFA, docosatetraenoic acid (ADA, 22:4n-6) which is also called adrenic acid. ADA is the third most abundant LCPUFA in the brain, and it is enriched in myelin lipids, especially in phosphatidylethanolamine (PE). Adrenic acid also accumulates rapidly during the early post-natal period of brain development in infants. It is clear that in order to meet the rapid production of adrenic acid needed for neural tissue development in infants, the conversion of ARA to adrenic acid is paramount (Hadley et al. 2016; Tounian et al. 2021). Dietary sources of ARA include lean red meat and chicken, egg yolks and human milk (Bradbury, 2011).

Infants in their first year of life undergo a phase of immune immaturity, making them more vulnerable to infections. This also is the most important period of T-cell differentiation and tolerance development to environmental and dietary antigens and is typically characterized by a sharp increase in ARA accumulation in the thymus (Hadley et al. 2016; Tounian et al. 2021). Evidence suggests that ARA plays a key immunoregulatory role and could be involved in the prevention of infections and allergic diseases.

Iron is another essential element present in every living organism and is a key mineral in human metabolism. Iron exists in two principal oxidation states, the ferrous (Fe²⁺) and the ferric (Fe³⁺) due to its capability of accepting and donating electrons and participating in oxidation-reduction (redox) reactions (Abbaspour et al. 2014; Briguglio et al. 2020; Eussen et al. 2015; Aisen et al. 2001). The biochemical functions of iron and their roles in diverse cellular metabolic processes depend on the redox states of iron (Aisen et al. 2001; Lieu et al. 2001). The most stable state of iron in biological complexes at physiological oxygen concentrations is Fe³⁺ (Aisen et al. 2001). Transmembrane transport of iron, iron deposition in ferritin (the storage protein) and heme synthesis all make use of Fe²⁺, underscoring the critical role of reduction reactions in iron metabolism (Aisen et al. 2001).

Accordingly, there is a need for a novel nutritional formula for infants and toddlers, including those that provide increased levels/bioavailability of key nutrients such as those discussed above in order to promote a more desirable development in e.g., brain, eye and/or immune system. Aspects of this disclosure are directed to meeting these and other needs.

### SUMMARY OF THE DISCLOSURE

Nutritional formulas are important sources of key dietary components, including proteins, carbohydrates, fats, vitamins and minerals. According to certain aspects, nutritional formulas will have a precisely defined composition that is specifically tailored for a particular target group. For example, an infant formula can offer nutrition suitable for infants, while other formulas can provide important dietary supplementation for seniors and individuals who have special nutritional requirements.

Accordingly, one embodiment of the present disclosure is a nutritional formula for an infant or a toddler.

Another embodiment of the present disclosure is a method for promoting development in an infant or a toddler, such as development of the brain, eye and/or immune system. Aspects of the method comprise administering a nutritional formula disclosed herein to the infant or toddler.

### BRIEF DESCRIPTION OF THE DRAWINGS

To facilitate further description of the embodiments of this disclosure, the following drawings are provided to illustrate and not to limit the scope of the disclosure.

FIG. 1 shows the effect of the form of folate used for each formula in the plasma content of folic acid. The bottom panel shows the source of folate in each formula.

FIG. 2 shows the effect of the form of folate used for each formula in the plasma content of 5-MTHF. The bottom panel shows the source of iron in each formula.

FIG. 3 shows the effect of the form of iron used for each formula in the hemoglobin level.

FIG. 4 shows the effect of the form of iron used for each formula in the Total Iron Binding Capacity (TIBC).

FIG. 5 shows the effect of the form of iron used for each formula in the plasma content of iron.

### DETAILED DESCRIPTION OF THE DISCLOSURE

Aspects of the present disclosure provide a nutritional formula for an infant or a toddler. According to certain embodiments, the nutritional formula contains optimized levels of key nutrients that are critical for development of the infant or toddler, such as key nutrients critical to brain/eye/immune system development. According to other embodiments, the nutritional formula also offers increased absorption and/or digestive tolerance via more bioavailable and/or tolerable forms of such nutrients.

### Definitions

In some embodiments, the formula is for a preterm or full term infant. As used herein, a “preterm” birth refers to an alive birth before 37 weeks of pregnancy are completed. There are sub-categories of preterm birth, based on gestational age: extremely preterm (less than 28 weeks), very preterm (28 to 32 weeks), moderate to late preterm (32 to 37 weeks). In the context of this disclosure, a “term” birth refers to an alive birth anytime between 37 weeks and 42 weeks of pregnancy, and a “full term” birth is an alive birth during 39 weeks and 40 weeks of pregnancy.

The term “synergistic” as used herein refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-inflammatory; ingredient B is pro-inflammatory; when A and B are combined, said combination produces an anti-inflammatory effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

In the present disclosure, an “effective amount”; of an agent is an amount of such an agent that is sufficient to effect beneficial or desired results or desired physiologic effect as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other agents being administered, the age, size, and species of the subject, and like factors well known in the arts of, e.g., nutritional formula or supplementation. In general, a suitable dose of an agent according to the disclosure will be that amount of the agent, which is the lowest dose effective to produce the desired effect with no or minimal side effects.

Salts may include counterions selected from chloride, bromide, iodide, and the like. Further salts may include, but are not limited to, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, tartrate, malate, citrate, sulfonate, trifluoromethanesulfonate, trichloromethanesulfonate, tribromomethanesulfonate, and trifluoroacetate.

### DESCRIPTION OF EMBODIMENTS

Folic acid is the main source of supplemental folate in infant and toddler formulas. Folic acid does not occur in nature. Folic acid is primarily produced by chemical synthesis, although biosynthetic approaches have also been attempted (Serrano-Amatriain et al. 2016). Several conversion steps are necessary to convert folic acid into L-5 methyl tetrahydrofolate (L-5-MTHF), the active form of folate. One of these steps is catalyzed by methylenetetrahydrofolate reductase (MTHFR) (Shane, 2008). The two common mutations of the MTHFR gene, namely C677T, A1298C have been proposed for an association with various pathological conditions and it also affects how an individual is able to efficiently transform folic acid into L-5-MTHF (Rosenblatt, 1995; Weisberg et al. 1998). The majority of folate in breast milk is in the form of L-5-MTHF and breastfeeding infants are exposed to L-5-MTHF from birth (Troesch et al. 2019; Büttner et al. 2013). Higher intakes of folic acid results in the presence of unmetabolized folic acid in the serum, the effects of which have yet to be elucidated. It has been suggested that infants homozygous for one or both polymorphisms would potentially benefit even more from the use of L-5-MTHF instead of folic acid in infant formula.

Accordingly, in some embodiments, the formula comprises an effective amount of L-methylfolate or its nutritionally acceptable salt. As used herein, a “nutritionally acceptable salt” refers to a salt form that is non-toxic and safe for administration to infants and toddlers in nutritional formulas and/or food supplements. Non-limiting examples of a nutritionally acceptable salt of L-methylfolate include calcium L-methylfolate, monosodium L-methylfolate, glucosamine L-methylfolate, magnesium L-methylfolate, or combinations thereof. In some embodiments, the nutritionally acceptable salt of L-methylfolate is calcium L-methylfolate.

In some embodiments, the formula comprises the L-methylfolate or its nutritionally acceptable salt in an amount of at least 4.5 μg/100 kcal, at least 7.5 μg/100 kcal, at least 10 μg/100 kcal, at least 15 μg/100 kcal, at least 20 μg/100 kcal, at least 25 μg/100 kcal, at least 30 μg/100 kcal, at least 35 μg/100 kcal, at least 40 μg/100 kcal, or at least 45 μg/100 kcal.

In some embodiments, the formula comprises the L-methylfolate or its nutritionally acceptable salt in an amount of no more than 48 μg/100 kcal, no more than 45 μg/100 kcal, no more than 40 μg/100 kcal, no more than 35 μg/100 kcal, no more than 30 μg/100 kcal, no more than 25 μg/100 kcal, no more than 20 μg/100 kcal, no more than 15 μg/100 kcal, no more than 10 μg/100 kcal, or no more than 7.5 μg/100 kcal.

In some embodiments, the formula comprises the L-methylfolate or its nutritionally acceptable salt in a range of about 4.5-48 μg/100 kcal. In some embodiments, the formula comprises the L-methylfolate or its nutritionally acceptable salt in a range of about 15.5-48 μg/100 kcal.

In one embodiment, the formula comprises a partial replacement of folic acid with L-methylfolate as a source of the vitamin, folate, such that L-methyl folate constitutes 25-99.9% by molar basis of the total folate label claim and folic acid constitutes 0.1-75% of the total folate label claim in the formula. In one embodiment, use levels will be equivalent on a molar basis to current folic acid use levels, to provide the same amount of folate. For example, in some embodiments, the formula comprises L-5 methyl tetrahydrofolate (L-5-MTHF) constituting 100% by molar basis of the total folate label claim. In some embodiments, the formula comprises L-5-MTHF constituting 90% by molar basis of the total folate label claim, and folic acid constituting 10% by molar basis of the total folate label claim. In some embodiments, the formula comprises L-5-MTHF constituting 80% by molar basis of the total folate label claim, and folic acid constituting 20% by molar basis of the total folate label claim. In some embodiments, the formula comprises L-5-MTHF constituting 70% by molar basis of the total folate label claim, and folic acid constituting 30% by molar basis of the total folate label claim. In some embodiments, the formula comprises L-5-MTHF constituting 60% by molar basis of the total folate label claim, and folic acid constituting 40% by molar basis of the total folate label claim. In some embodiments, the formula comprises L-5-MTHF constituting 50% by molar basis of the total folate label claim, and folic acid constituting 50% by molar basis of the total folate label claim. In some embodiments, the formula comprises L-5-MTHF constituting 40% by molar basis of the total folate label claim, and folic acid constituting 60% by molar basis of the total folate label claim. In some embodiments, the formula comprises L-5-MTHF constituting 30% by molar basis of the total folate label claim, and folic acid constituting 70% by molar basis of the total folate label claim. In some embodiments, the formula comprises L-5-MTHF constituting 20% by molar basis of the total folate label claim, and folic acid constituting 80% by molar basis of the total folate label claim. In some embodiments, the formula comprises L-5-MTHF constituting 10% by molar basis of the total folate label claim, and folic acid constituting 90% by molar basis of the total folate label claim.

It has been established that the first year of life is a critical time of growth and development; several organs and systems including the brain, retina, and immune system rapidly mature during this time. Recent scientific evidence demonstrates that dietary variation of DHA and ARA during the first few months of life may have long-term effects on cognitive function. In addition, it has been determined that dietary DHA and ARA are essential for visual development and acuity in term infants. Hence, providing adequate amounts of these important long chain polyunsaturated fatty acids (LCPUFAs) throughout infancy can impact optimal growth and development (Tai et al. 2013; Bradbury, 2011; Tounian et al. 2021; Lien et al. 2017).

Although most infants have the ability to synthesize LCPUFAs including DHA and ARA, the conversion rate may be insufficient to synthesize adequate amounts from their precursors to meet demands of their rapid tissue growth and some infants may not be even able to synthesize any DHA or ARA (Tai et al. 2013; Uauy et al. 2000) making them conditionally essential nutrients. About 80% of the total amounts of DHA and ARA present in neonatal tissues at term delivery are accumulated during the last trimester of pregnancy. Especially, preterm infants are at an even higher risk of LCPUFAs depletion than term infants as they are born with huge deficits of these fatty acids (Tai et al. 2013; Uauy et al. 2000). Very low birth weight infants are also not able to synthesize DHA and ARA in adequate amounts. Genetics plays a major role determining the rates of conversion from LA to ARA and ALA to DHA resulting in huge interindividual variations (Linda et al. 2006). Endogenous conversion of precursors into LCPUFAs is determined by the activities of the enzymes delta-5 desaturase and delta-6 desaturase and the conversion rate results from variations in the desaturase gene cluster (Linda et al. 2006). Therefore, supplementing DHA and ARA in the diet is necessary to newborns because of the limited endogenous synthesis.

Accordingly, in some embodiments, the formula comprises an effective amount of docosahexaenoic acid (DHA). In some embodiments, the formula comprises Schizochytrium oil as a source of DHA. Other sources of DHA may include, for example, fish oil.

In some embodiments, the formula comprises at least 21 mg/100 kcal, at least 23 mg/100 kcal, at least 25 mg/100 kcal, at least 30 mg/100 kcal, at least 35 mg/100 kcal, at least 40 mg/100 kcal, at least 45 mg/100 kcal, at least 50 mg/100 kcal, or at least 55 mg/100 kcal of DHA.

In some embodiments, the formula comprises no more than 60 mg/100 kcal, no more than 55 mg/100 kcal, no more than 50 mg/100 kcal, no more than 45 mg/100 kcal, no more than 40 mg/100 kcal, no more than 35 mg/100 kcal, no more than 30 mg/100 kcal, no more than 25 mg/100 kcal, or no more than 23 mg/100 kcal of DHA.

In some embodiments, the formula comprises DHA in a range of about 21-60 mg/100 kcal. In some embodiments, the formula comprises DHA in a range of about 23-40 mg/100 kcal.

In some embodiments, the formula comprises L-methylfolate or its nutritionally acceptable salt and DHA at a wt./100 kcal ratio in a range of from about 1:11000 to about 1:209.

Accumulating clinical evidence indicates that DHA and ARA supplementation instead of DHA alone may be more efficacious for optimal cognitive development (Hadley et al. 2016; Lien et al. 2017). In a clinical study, infants receiving both DHA and ARA showed a comparable rate of vocabulary development similar to breastfed infants at 14 months, whereas infants receiving DHA alone scored lower in some aspects of vocabulary development (Scott et al. 1998). Another randomized controlled trial showed that a formula containing DHA alone had no significant benefits in cognitive development, whereas a combination of DHA and ARA supplementation during the first 4 months resulted in a mean increase of 7 points on the Major Depression Inventory score when the children were evaluated at 18 months of age (Birth et al. 2000).

Accordingly, in some embodiments, the formula comprises an effective amount of arachidonic acid (ARA). In some embodiments, the formula comprises Mortierella alpina oil as a source of ARA. Other sources of ARA may include, for example, fish oil.

In some embodiments, the formula comprises at least 35 mg/100 kcal, at least 40 mg/100 kcal, at least 45 mg/100 kcal, at least 50 mg/100 kcal, at least 55 mg/100 kcal, at least 60 mg/100 kcal, at least 65 mg/100 kcal, at least 70 mg/100 kcal, at least 75 mg/100 kcal, at least 80 mg/100 kcal, at least 85 mg/100 kcal, at least 90 mg/100 kcal, at least 95 mg/100 kcal, at least 100 mg/100 kcal, at least 105 mg/100 kcal, at least 110 mg/100 kcal, or at least 115 mg/100 kcal of ARA.

In some embodiments, the formula comprises no more than 120 mg/100 kcal, no more than 115 mg/100 kcal, no more than 110 mg/100 kcal, no more than 105 mg/100 kcal, no more than 100 mg/100 kcal, no more than 95 mg/100 kcal, no more than 90 mg/100 kcal, no more than 85 mg/100 kcal, no more than 80 mg/100 kcal, no more than 75 mg/100 kcal, no more than 70 mg/100 kcal, no more than 65 mg/100 kcal, no more than 60 mg/100 kcal, no more than 55 mg/100 kcal, no more than 50 mg/100 kcal, no more than 45 mg/100 kcal, or no more than 40 mg/100 kcal of ARA.

In some embodiments, the formula comprises ARA in a range of about 35-120 mg/100 kcal. In some embodiments, the formula comprises ARA in a range of about 35-80 mg/100 kcal.

In some embodiments, the formula comprises DHA and ARA at a wt./100 kcal ratio in a range of from about 1:1 to about 1:2.

Iron deficiency is the most common single-nutrient deficiency among children in the developing world (Baker and Greer, 2010; Sherry et al. 2001). About 80% of the iron found in a newborn term infant is accumulated during the third trimester of pregnancy. Various factors such as maternal conditions, anemia, maternal hypertension, or diabetes during pregnancy have been suggested to result in low fetal iron stores in both, term and preterm infants (Baker and Greer, 2010). Preterm infants miss rapid accretion of iron in the third trimester of pregnancy and are therefore deficient in total body iron and this deficit increases with decreasing gestational age (Baker and Greer, 2010). This is further exacerbated by the rapid postnatal growth that many infants experience and by frequent phlebotomies without adequate blood replacement. In contrast, preterm infants who receive multiple blood transfusions are susceptible to the risk of iron overload (Baker and Greer, 2010).

Infants born at term usually have sufficient iron stores until 4 to 6 months of age. These infants have high hemoglobin concentration and high blood volume in proportion to their body weight but they also experience a physiologic decline in both blood volume and hemoglobin concentration during the first several months of life (Baker and Greer, 2010; Dallman, 1993). For these reasons it is generally thought that breastfed infants need very little iron since the small amount of iron in human milk is assumed to be sufficient for the exclusively breastfed infant (Baker and Greer, 2010). However, given the large variation in human milk iron content and the fact that bigger infants may ingest more milk, the amount of iron provided by maternal milk may not meet the demands of the infant (Baker and Greer, 2010). Increased risk of iron deficiency anemia at 9 months of age has been associated with exclusive breastfeeding for more than 6 months (Dallman, 1993; Meinzen-Derr et al. 2006). Also, infants who are born with lower than usual iron stores (low birth weight infants, infants of diabetic mothers), a condition linked to lower serum ferritin levels at 9 months of age are not taken into account in the recommendations for exclusive breastfeeding for 6 months (Georgieff et al. 2002). Friel et al showed that exclusively breastfed infants supplemented with iron between 1 and 6 months of age exhibited higher hemoglobin levels and higher mean corpuscular volume at 6 months of age than did their unsupplemented counterparts (Friel et al. 2003). In addition, better visual acuity and higher Bayley Psychomotor Developmental Indices were also observed at 13 months owing to the supplementation (Friel et al. 2003).

Accordingly, in some embodiments, the formula comprises an effective amount of iron salt as a source of elemental iron. In the present disclosure, more bioavailable and tolerable forms of iron are provided. For example, in some embodiments, the iron salt is selected from the group consisting of iron (III) pyrophosphate (ferric pyrophosphate), iron (II) fumarate (ferrous fumarate), iron (II) gluconate (ferrous gluconate), iron (II) sulfate (ferrous sulfate), iron (II) bisglycinate (ferrous bisglycinate chelate), or combinations thereof.

In some embodiments, the formula comprises at least 1.1 mg/100 kcal, at least 1.2 mg/100 kcal, at least 1.3 mg/100 kcal, at least 1.4 mg/100 kcal, or at least 1.5 mg/100 kcal of the iron salt as elemental iron.

In some embodiments, the formula comprises no more than 1.6 mg/100 kcal, no more than 1.5 mg/100 kcal, no more than 1.4 mg/100 kcal, no more than 1.3 mg/100 kcal, or no more than 1.2 mg/100 kcal of the iron salt as elemental iron.

In some embodiments, the formula comprises the iron salt in a range of about 1.1-1.6 mg/100 kcal as elemental iron. In some embodiments, the formula comprises the iron salt in a range of about 1.3-1.4 mg/100 kcal as elemental iron.

In some embodiments, the formula comprises ferric pyrophosphate constituting 100% by molar basis of the total elemental iron label claim. In some embodiments, the formula comprises ferric pyrophosphate constituting 90% by molar basis of the total elemental iron label claim, and another iron form constituting 10% by molar basis of the total elemental iron label claim. In some embodiments, the formula comprises ferric pyrophosphate constituting 80% by molar basis of the total elemental iron label claim, and another iron form constituting 20% by molar basis of the total elemental iron label claim. In some embodiments, the formula comprises ferric pyrophosphate constituting 70% by molar basis of the total elemental iron label claim, and another iron form constituting 30% by molar basis of the total elemental iron label claim. In some embodiments, the formula comprises ferric pyrophosphate constituting 60% by molar basis of the total elemental iron label claim, and another iron form constituting 40% by molar basis of the total elemental iron label claim. In some embodiments, the formula comprises ferric pyrophosphate constituting 50% by molar basis of the total elemental iron label claim, and another iron form constituting 50% by molar basis of the total elemental iron label claim. In some embodiments, the formula comprises ferric pyrophosphate constituting 40% by molar basis of the total elemental iron label claim, and another iron form constituting 60% by molar basis of the total elemental iron label claim. In some embodiments, the formula comprises ferric pyrophosphate constituting 30% by molar basis of the total elemental iron label claim, and another iron form constituting 70% by molar basis of the total elemental iron label claim. In some embodiments, the formula comprises ferric pyrophosphate constituting 20% by molar basis of the total elemental iron label claim, and another iron form constituting 80% by molar basis of the total elemental iron label claim. In some embodiments, the formula comprises ferric pyrophosphate constituting 10% by molar basis of the total elemental iron label claim, and another iron form constituting 90% by molar basis of the total elemental iron label claim. In some embodiments, the another iron form is selected from ferrous sulfate, ferrous bisglycinate chelate, or combinations thereof.

In some embodiments, the formula comprises L-methylfolate or its nutritionally acceptable salt and iron salt at a wt./100 kcal ratio in a range of from about 1:750 to about 1:3. In some embodiments, the formula comprises L-methylfolate or its nutritionally acceptable salt and iron salt at a wt./100 kcal ratio in a range of from about 1:222 to about 1:20.

In some embodiments, the formula comprises ferric pyrophosphate constituting 100% by molar basis of the total elemental iron label claim, and L-5-MTHF constituting 100% by molar basis of the total folate label claim. In some embodiments, the formula comprises ferric pyrophosphate constituting 100% by molar basis of the total elemental iron label claim, and L-5-MTHF constituting 50% by molar basis of the total folate label claim and folic acid constituting 50% by molar basis of the total folate label claim. In some embodiments, the formula comprises ferric pyrophosphate constituting 50% by molar basis of the total elemental iron label claim and ferrous sulfate constituting 50% by molar basis of the total elemental iron label claim, and L-5-MTHF constituting 100% by molar basis of the total folate label claim. In some embodiments, the formula comprises ferric pyrophosphate constituting 50% by molar basis of the total elemental iron label claim and ferrous sulfate constituting 50% by molar basis of the total elemental iron label claim, and L-5-MTHF constituting 50% by molar basis of the total folate label claim and folic acid constituting 50% by molar basis of the total folate label claim.

In some embodiments, the formula comprises iron salt and DHA at a wt./100 kcal ratio in a range of from about 1:333 to about 1:3.

Vitamin B12 (B12, cobalamin) is important during early development, and the newborn child depends on breast milk or infant formula for supply of the vitamin. Vitamin B12 supplements are typically derived from two sources: cyanocobalamin or methylcobalamin. Cyanocobalamin is a synthetic form of vitamin B12 that is not found in nature, while methylcobalamin is a naturally occurring form of vitamin B12 that can be obtained through supplements, as well as food sources like fish, meat, eggs, and milk.

In some embodiments of the present disclosure, the formula comprises an effective amount of methylcobalamin as a source of vitamin B12.

In some embodiments, the formula comprises at least 0.23 μg/100 kcal, at least 0.25 μg/100 kcal, at least 0.3 μg/100 kcal, at least 0.35 μg/100 kcal, at least 0.4 μg/100 kcal, or at least 0.45 μg/100 kcal of methylcobalamin.

In some embodiments, the formula comprises no more than 0.5 μg/100 kcal, no more than 0.45 μg/100 kcal, no more than 0.4 μg/100 kcal, no more than 0.35 μg/100 kcal, no more than 0.3 μg/100 kcal, or no more than 0.25 μg/100 kcal of methylcobalamin.

In some embodiments, the formula comprises methylcobalamin in a range of about 0.23-0.5 μg/100 kcal. In some embodiments, the formula comprises methylcobalamin in a range of about 0.25-0.35 μg/100 kcal.

Other than the ingredients mentioned above, a typical nutritional formula often comprises various other required nutrients/supplements. For example, in some embodiments, the formula further comprises an effective amount of at least one of ascorbic acid, cholecalciferol, L-carnitine-L-tartrate, betaine, taurine, or nutritionally acceptable salts thereof, or combinations thereof.

In some embodiments, the formula comprises at least 4 mg/100 kcal, at least 5 mg/100 kcal, at least 8 mg/100 kcal, at least 10 mg/100 kcal, at least 15 mg/100 kcal, at least 20 mg/100 kcal, or at least 25 mg/100 kcal of ascorbic acid or its nutritionally acceptable salt.

In some embodiments, the formula comprises no more than 30 mg/100 kcal, no more than 25 mg/100 kcal, no more than 20 mg/100 kcal, no more than 15 mg/100 kcal, no more than 10 mg/100 kcal, no more than 8 mg/100 kcal, or no more than 5 mg/100 kcal of ascorbic acid or its nutritionally acceptable salt.

In some embodiments, the formula comprises ascorbic acid or its nutritionally acceptable salt in a range of about 4-30 mg/100 kcal. In some embodiments, the formula comprises ascorbic acid or its nutritionally acceptable salt in a range of about 8-30 mg/100 kcal.

In some embodiments, the formula comprises cholecalciferol in a range of about 1-3 μg/100 kcal. In some embodiments, the formula comprises cholecalciferol in a range of about 2-2.5 μg/100 kcal.

In some embodiments, the formula comprises at least 1.2 mg/100 kcal, at least 1.5 mg/100 kcal, at least 2 mg/100 kcal, at least 2.5 mg/100 kcal, at least 3 mg/100 kcal, at least 3.5 mg/100 kcal, at least 4 mg/100 kcal, or at least 4.5 mg/100 kcal of L-carnitine-L-tartrate.

In some embodiments, the formula comprises no more than 5 mg/100 kcal, no more than 4.5 mg/100 kcal, no more than 4 mg/100 kcal, no more than 3.5 mg/100 kcal, no more than 3 mg/100 kcal, no more than 2.5 mg/100 kcal, no more than 2 mg/100 kcal, or no more than 1.5 mg/100 kcal of L-carnitine-L-tartrate.

In some embodiments, the formula comprises L-carnitine-L-tartrate in a range of about 1.2-5 mg/100 kcal. In some embodiments, the formula comprises L-carnitine-L-tartrate in a range of about 1.5-3 mg/100 kcal.

In some embodiments, the formula comprises L-carnitine-L-tartrate and DHA at a wt./100 kcal ratio in a range of from about 1:42 to about 1:2.

In some embodiments, the formula comprises L-carnitine-L-tartrate and iron salt at a wt./100 kcal ratio in a range of from about 1:2.5 to about 33:1.

In some embodiments, the formula comprises L-carnitine-L-tartrate and betaine at a wt./100 kcal ratio in a range of from about 1:10 to about 5:1.

In some embodiments, the formula comprises L-carnitine-L-tartrate and taurine at a wt./100 kcal ratio in a range of from about 1:10 to about 5:1.

In some embodiments, the formula comprises L-carnitine-L-tartrate and L-methylfolate or its nutritionally acceptable salt at a wt./100 kcal ratio in a range of from about 1000:1 to about 25:1.

In some embodiments, the formula further comprises at least one of soy lecithin, canola lecithin, sunflower lecithin, calcium phosphate, potassium citrate, potassium phosphate, sodium chloride, sodium citrate, calcium carbonate, potassium hydroxide, potassium phosphate, magnesium chloride, potassium bicarbonate, potassium chloride, zinc sulfate, cupric sulfate, manganese sulfate, potassium iodide, sodium selenite, choline bitartrate, choline chloride, ascorbic acid, ascorbyl palmitate, inositol, mixed tocopherol concentrate, dl-alpha tocopheryl acetate (vitamin E), niacinamide (vitamin B3), calcium pantothenate, vitamin A palmitate, vitamin A acetate, riboflavin (vitamin B2), thiamine hydrochloride (vitamin B1), pyridoxine hydrochloride (vitamin B6), folic acid, phytonadione (vitamin K), biotin, cholecalciferol (vitamin D3), cyanocobalamin (vitamin B12), or combinations thereof.

Additional component included in the formula of the present disclosure is protein, a critical nutrient for growth, synthesis of enzymes and hormones. The proteins, as used herein, may include any proteins or nitrogen source suitable for human, especially infant, consumption. Such proteins are well known in the art and can be readily selected and prepared. Non-limiting examples of suitable protein sources include casein, whey, condensed skim milk, nonfat milk, soy, pea, rice, corn, hydrolyzed protein, free amino acids, or combinations thereof. For example, in some embodiments, the formula is a dairy-based formula. In some embodiments, the milk source is cow milk, which can be skimmed milk or whole milk. In other embodiments, the milk source can from goats, which can be skimmed goat milk or whole goat milk. In yet another embodiment, the protein source is a soy or vegetable-based protein source. According to yet another embodiment, the source of protein comprises at least partially or even fully hydrolyzed protein, such as hydrolyzed caseinates, hydrolyzed whey, or other hydrolyzed milk proteins, and/or hydrolyzed soy or other vegetable proteins, and may even comprise protein that has been hydrolyzed down to its constituent amino acids. Commercial protein sources are readily available and known to those skilled in the art.

A person skilled in the art would appreciate that the formula of the present disclosure can be in any form suitable for any standard use in the food industry. For example, in one embodiment, the formula is in powdered form that has a long shelf life and is convenient for storage and transportation. In another embodiment, the formula is in liquid form that is packaged for instant use, such as a reconstituted liquid form.

Another embodiment of the present disclosure is a method for promoting brain, eye and/or immune system development in an infant or a toddler. This method comprises administering a nutrient formula disclosed herein to the infant or toddler.

In some embodiments, the method comprises administering a nutrient formula disclosed herein to an infant that is preterm.

In the present disclosure, an “effective amount” of an agent is an amount of such an agent that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other agents being administered, the age, size, and species of the subject, and like factors well known in the arts of, e.g., nutritional formula or supplementation. In general, a suitable dose of an agent according to the disclosure will be that amount of the agent, which is the lowest dose effective to produce the desired effect with no or minimal side effects.

The following example is provided to further illustrate the formula of the present disclosure. This example is illustrative only and is not intended to limit the scope of the disclosure in any way.

### Prophetic Example

Certain hypothesis with respect to the effects and possible synergy of components of formula embodiments disclosed herein will be tested. For example, without being limited to any particular theory, it is believed that L-methylfolate will be absorbed more efficiently than other forms of folate, and will be more readily available to participate in methylation pathways than other forms of folate. It is also believed that, in the absence of nucleotides, L-methylfolate will support the efficient synthesis of DNA, thus of supporting the immune system. It is further believed that L-methylfolate will be more efficiently metabolized that folic acid, thus resulting in lower serum levels of unmetabolized folate. To test these hypotheses, animal experiments will performed to determine whether L-methylfolate and other bioactive ingredients in the formula according to aspects of the present disclosure have effects on parameters such as lipid absorption, immune parameters (e.g., cytokines), fermentation-generated metabolites (e.g., short chain fatty acids), iron absorption, and generation of unmetabolized serum folate.

An example of an experiment protocol is as below.

| Nutrient | Control | Test Formula 1 | Test Formula 2 | Sow Fed |
|---|---|---|---|---|
| DHA | 20 mg/100 kcal | 30 mg/100 kcal | 30 mg/100 kcal | n/a |
| ARA | 34 mg/100 kcal | 51 mg/100 kcal | 51 mg/100 kcal | n/a |
| DHA:ARA | 1:1.7 | 1:1.7 | 1:1.7 | |
| Folic Acid | 15 mcg/100 kcal | 0 | 7.5 mcg/100 kcal | n/a |
| L-Methylfolate | 0 | 18 mcg/100 kcal | 8.5 mcg/100 kcal | n/a |
| Folic Acid:L- | | | 1:1.3 | |
| Methylfolate | | | | |
| Elemental iron | 1.2 mg/100 kcal (as | 1.2 mg/100 kcal (as | 1.2 mg/100 kcal (as | n/a |
| | ferrous sulfate) | ferrous gluconate) | ferrous gluconate) | |

Animals are to be assigned to four groups according to the different formulae administered to each, namely a control group, test formula 1, test formula 2, and a sow fed group. Each group will have seven piglets. The blood, stool and organs of each piglet will be collected and tested for the overall growth parameters, nutrient concentration levels, and unmetabolized folate levels. The results within the same group will be normalized and then compared across the groups to assess overall results.

### Example 2

Certain hypothesis with respect to the effects and possible synergy of components of formula embodiments disclosed herein were tested. For example, without being limited to any particular theory, it is believed that L-methylfolate will be absorbed more efficiently than other forms of folate, and will be more readily available to participate in methylation pathways than other forms of folate. It is also believed that, in the absence of nucleotides, L-methylfolate will support the efficient synthesis of DNA, thus of supporting the immune system. It is further believed that L-methylfolate will be more efficiently metabolized that folic acid, thus resulting in lower serum levels of unmetabolized folate. To test these hypotheses, animal experiments were performed to determine whether L-methylfolate and other bioactive ingredients (e.g., elemental iron) in the formula according to aspects of the present disclosure have effects on parameters such as lipid absorption, immune parameters (e.g., cytokines), fermentation-generated metabolites (e.g., short chain fatty acids), iron absorption, and generation of unmetabolized serum folate.

The experimental protocol is as below.

TABLE 1
Ingredients and nutrient composition of the experimental formulas.

| | | | | | Sow- |
|---|---|---|---|---|---|
| Ingredients, % | CON | FOR 1 | FOR 2 | FOR 3 | reared |
| Water | 83.008 | 83.008 | 83.008 | 83.008 | n/a |
| Whey protein isolate¹ | 6.162 | 6.162 | 6.162 | 6.162 | n/a |
| Corn oil | 4.704 | 4.645 | 4.645 | 4.645 | n/a |
| Lactose² | 3.000 | 3.000 | 3.000 | 3.000 | n/a |
| Trace mineral premix³ | 1.125 | 1.125 | 1.125 | 1.125 | n/a |
| Casein² | 1.073 | 1.073 | 1.073 | 1.073 | n/a |
| Sodium phosphate, | 0.379 | 0.379 | 0.379 | 0.379 | n/a |
| dibasic | | | | | |
| Vitamin premix³ | 0.250 | 0.250 | 0.250 | 0.250 | n/a |
| DHA:ARA Blend⁴ | 0.107 | 0.166 | 0.166 | 0.166 | n/a |
| Xanthum gum | 0.100 | 0.100 | 0.100 | 0.100 | n/a |
| Calcium carbonate | 0.092 | 0.092 | 0.092 | 0.092 | n/a |

Calculated Analysis

| | | | | | |
|---|---|---|---|---|---|
| Protein, % | 6.5 | 6.5 | 6.5 | 6.5 | n/a |
| Fat, % | 4.6 | 4.6 | 4.6 | 4.6 | n/a |
| Lactose, % | 3.0 | 3.0 | 3.0 | 3.0 | n/a |
| Calcium, % | 0.23 | 0.23 | 0.23 | 0.23 | n/a |
| Phosphorus, % | 0.17 | 0.17 | 0.17 | 0.17 | n/a |
| Metabolizable Energy | 820 | 820 | 820 | 820 | n/a |
| (kcal/kg) | | | | | |

¹Milk Specialties (Eden Prairie, MN)
²Bulk Foods Direct Nutrition (Tempe, AZ)
³Dyets Inc (Bethlehem, PA). Vitamin premix provided (g/kg): thiamine HCl, 0.1; riboflavin, 0.375; pyridoxine HCl, 0.1; niacin, 1; calcium pantothenate, 1.2; No folic acid, 0.13; biotin, 0.02; cobalamin, 1.5; retinyl palmitate, 0.8; cholecalciferol, 0.05; tocopheryl acetate, 8.8; menadione sodium bisulfate, 0.08. Trace mineral premix provided (g/kg): calcium phosphate, dibasic, 187; calcium carbonate, 279; sodium chloride, 85; potassium phosphate monobasic, 155; magnesium sulfate, anhydrous, 44; manganous carbonate, 0.93; No ferric citrate, 10; zinc carbonate, 1.84; cupric carbonate, 0.193; potassium iodate, 0.005; sodium selenite, 0.007. To the basal vitamin and mineral premixes ferrous sulfate, Ferrous Bisglycinate chelate, Ferric pyrophosphate, Folic Acid, and 5-methyl terahydrofolate were added to create 4 premixes that were used to generate the experimental formulas (see Table 2).
⁴Bobbie Baby Inc.

TABLE 2
Supplemental ingredients added to
the mineral and vitamin premixes.

| | | | | | |
|---|---|---|---|---|---|
| | | | | | Sow- |
| Ingredient | CON | FOR 1 | FOR 2 | FOR 3 | reared |

g/kg mineral premix
Mineral premix

| | | | | | |
|---|---|---|---|---|---|
| Ferrous sulfate | 6.045 | 3.666 | 3.666 | 3.666 | n/a |
| Ferrous bisglycinate | — | 3.226 | — | — | n/a |
| chelate | | | | | |
| Ferric pyrophosphate | — | — | 11.672 | 11.672 | n/a |

Vitamin premix

| | | | | | |
|---|---|---|---|---|---|
| Folic acid | 0.4920 | — | 0.2460 | — | n/a |
| 5-methyl terahydrofolate | — | 0.0642 | 0.0303 | 0.0642 | n/a |

Animals were assigned to five groups according to the different formulae administered to each, namely a control group (CON), test formula 1 (FOR 1), test formula 2 (FOR 2), test formula 3 (FOR 3), and a sow-reared group. Each group had seven piglets. The blood, stool and organs of each piglet were collected and tested at Day 0, Day 7, Day 14 and Day 21 for the overall growth parameters, nutrient concentration levels, and unmetabolized folate levels. The results within the same group were normalized and then compared across the groups to assess overall results.

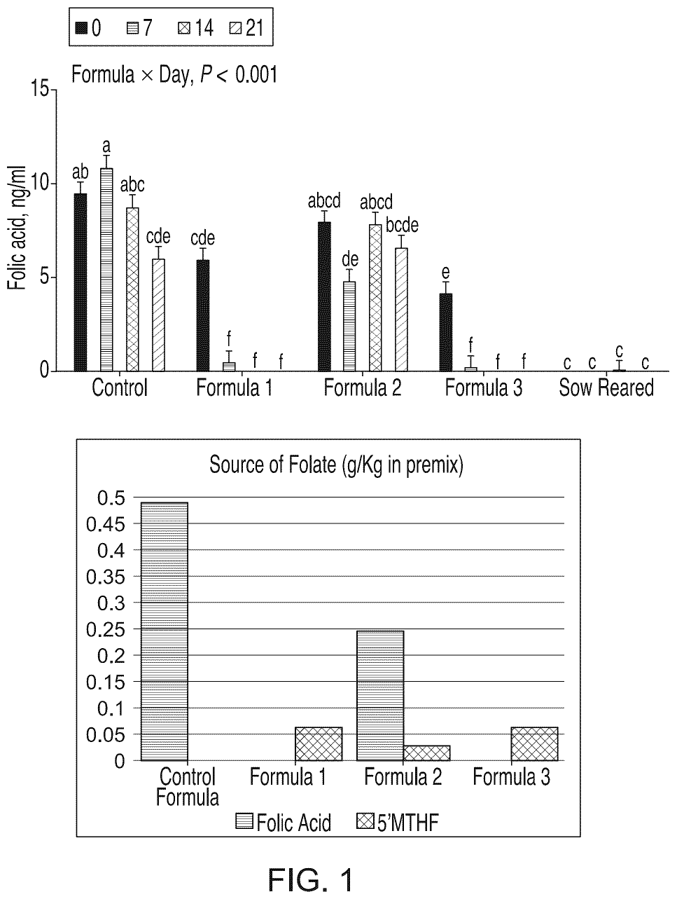

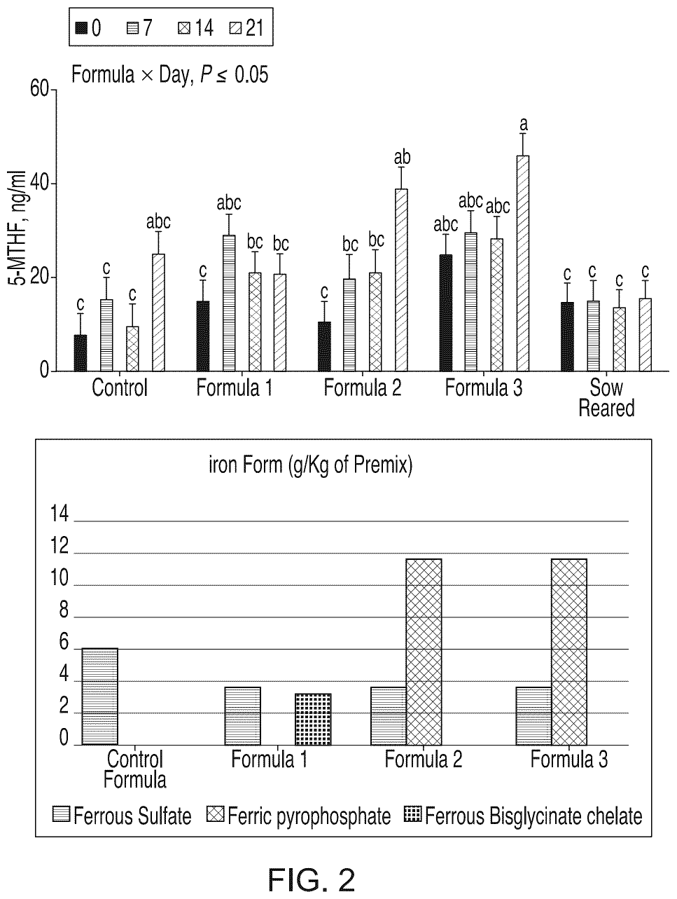

While FOR 1 and FOR 3 (which contains only 5-MTHF) resulted in undetectable plasma folic acid by Day 14 (FIG. 1), FOR 3 generated higher plasma concentrations of 5-MTHF than FOR 1 even though, both formulas have the same concentration of 5-MTHF (FIG. 2). Notably, FOR 2 has half the content of 5-MTHF yet it surpassed plasma content of 5-MTHF of the control group, FOR 1, and the sow-reared, which was comparable to that of FOR 3 (FIG. 2).

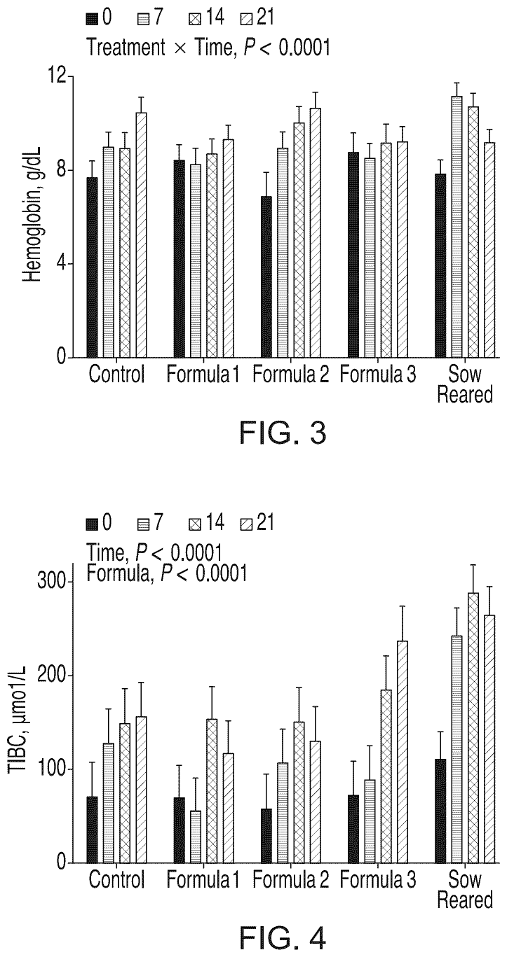

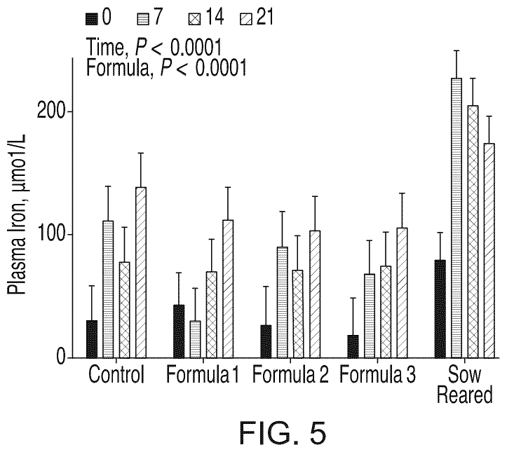

Referring to FIGS. 3-5, by substituting half of Ferrous sulfate with Ferric pyrophosphate as the source of elemental iron, FOR 2 caused an increase in hemoglobin through time (FIG. 3). FOR 3, on the other hand, different from FOR 2 by having only 5-MTHF in the vitamin premixture, caused an increase in Total Iron Binding Capacity like sow-reared and higher than all formulas at day 21 (FIG. 4), while plasma iron levels were similar in all formulas (FIG. 5).

The following embodiments are provided to illustrate aspects of the disclosure, although the embodiments are not intended to be limiting and other aspects and/or embodiments may also be provided.

Embodiment 1. A nutritional formula for an infant or a toddler.

Embodiment 2. The formula according to Embodiment 1, wherein the formula is for a preterm or full term infant.

Embodiment 3. The formula according to any preceding Embodiment, comprising an effective amount of L-methylfolate or its nutritionally acceptable salt.

Embodiment 4. The formula according to Embodiment 3, wherein the nutritionally acceptable salt of L-methylfolate is selected from the group consisting of calcium L-methylfolate, monosodium L-methylfolate, glucosamine L-methylfolate, magnesium L-methylfolate, or combinations thereof.

Embodiment 5. The formula according to Embodiment 3, wherein the nutritionally acceptable salt of L-methylfolate is calcium L-methylfolate.

Embodiment 6. The formula according to any one of Embodiments 3-5, wherein the formula comprises the L-methylfolate or its nutritionally acceptable salt in an amount of at least 4.5 μg/100 kcal, at least 7.5 μg/100 kcal, at least 10 μg/100 kcal, at least 15 μg/100 kcal, at least 20 μg/100 kcal, at least 25 μg/100 kcal, at least 30 μg/100 kcal, at least 35 μg/100 kcal, at least 40 μg/100 kcal, or at least 45 μg/100 kcal.

Embodiment 7. The formula according to any one of Embodiments 3-6, wherein the formula comprises the L-methylfolate or its nutritionally acceptable salt in an amount of no more than 48 μg/100 kcal, no more than 45 μg/100 kcal, no more than 40 μg/100 kcal, no more than 35 μg/100 kcal, no more than 30 μg/100 kcal, no more than 25 μg/100 kcal, no more than 20 μg/100 kcal, no more than 15 μg/100 kcal, no more than 10 μg/100 kcal, or no more than 7.5 μg/100 kcal.

Embodiment 8. The formula according to any one of Embodiments 3-7, wherein the formula comprises the L-methylfolate or its nutritionally acceptable salt in a range of about 4.5-48 μg/100 kcal.

Embodiment 9. The formula according to any one of Embodiments 3-7, wherein the formula comprises the L-methylfolate or its nutritionally acceptable salt in a range of about 15.5-48 μg/100 kcal.

Embodiment 10. The formula according to any preceding Embodiment, comprising an effective amount of docosahexaenoic acid (DHA).

Embodiment 11. The formula according to Embodiment 10, wherein the formula comprises Schizochytrium oil as a source of DHA.

Embodiment 12. The formula according to any one of Embodiments 10-11, wherein the formula comprises at least 21 mg/100 kcal, at least 23 mg/100 kcal, at least 25 mg/100 kcal, at least 30 mg/100 kcal, at least 35 mg/100 kcal, at least 40 mg/100 kcal, at least 45 mg/100 kcal, at least 50 mg/100 kcal, or at least 55 mg/100 kcal of DHA.

Embodiment 13. The formula according to any one of Embodiments 10-12, wherein the formula comprises no more than 60 mg/100 kcal, no more than 55 mg/100 kcal, no more than 50 mg/100 kcal, no more than 45 mg/100 kcal, no more than 40 mg/100 kcal, no more than 35 mg/100 kcal, no more than 30 mg/100 kcal, no more than 25 mg/100 kcal, or no more than 23 mg/100 kcal of DHA.

Embodiment 14. The formula according to any one of Embodiments 10-13, wherein the formula comprises DHA in a range of about 21-60 mg/100 kcal.

Embodiment 15. The formula according to any one of Embodiments 10-14, wherein the formula comprises DHA in a range of about 23-40 mg/100 kcal.

Embodiment 16. The formula according to any one of Embodiments 10-15, wherein the formula comprises L-methylfolate or its nutritionally acceptable salt and DHA at a wt./100 kcal ratio in a range of from about 1:11000 to about 1:209.

Embodiment 17. The formula according to any preceding Embodiment, comprising an effective amount of arachidonic acid (ARA).

Embodiment 18. The formula according to Embodiment 17, wherein the formula comprises Mortierella alpina oil as a source of ARA.

Embodiment 19. The formula according to any one of Embodiments 17-18, wherein the formula comprises at least 35 mg/100 kcal, at least 40 mg/100 kcal, at least 45 mg/100 kcal, at least 50 mg/100 kcal, at least 55 mg/100 kcal, at least 60 mg/100 kcal, at least 65 mg/100 kcal, at least 70 mg/100 kcal, at least 75 mg/100 kcal, at least 80 mg/100 kcal, at least 85 mg/100 kcal, at least 90 mg/100 kcal, at least 95 mg/100 kcal, at least 100 mg/100 kcal, at least 105 mg/100 kcal, at least 110 mg/100 kcal, or at least 115 mg/100 kcal of ARA.

Embodiment 20. The formula according to any one of Embodiments 17-19, wherein the formula comprises no more than 120 mg/100 kcal, no more than 115 mg/100 kcal, no more than 110 mg/100 kcal, no more than 105 mg/100 kcal, no more than 100 mg/100 kcal, no more than 95 mg/100 kcal, no more than 90 mg/100 kcal, no more than 85 mg/100 kcal, no more than 80 mg/100 kcal, no more than 75 mg/100 kcal, no more than 70 mg/100 kcal, no more than 65 mg/100 kcal, no more than 60 mg/100 kcal, no more than 55 mg/100 kcal, no more than 50 mg/100 kcal, no more than 45 mg/100 kcal, or no more than 40 mg/100 kcal of ARA.

Embodiment 21. The formula according to any one of Embodiments 17-20, wherein the formula comprises ARA in a range of about 35-120 mg/100 kcal.

Embodiment 22. The formula according to any one of Embodiments 17-21, wherein the formula comprises ARA in a range of about 35-80 mg/100 kcal.

Embodiment 23. The formula according to any one of Embodiments 17-22, wherein the formula comprises DHA and ARA at a wt./100 kcal ratio in a range of from about 1:1 to about 1:2.

Embodiment 24. The formula according to any preceding Embodiment, comprising an effective amount of iron salt as a source of elemental iron.

Embodiment 25. The formula according to Embodiment 24, wherein the iron salt is selected from the group consisting of iron (III) pyrophosphate (ferric pyrophosphate), iron (II) fumarate (ferrous fumarate), iron (II) gluconate (ferrous gluconate), iron (II) sulfate (ferrous sulfate), iron (II) bisglycinate (ferrous bisglycinate chelate), or combinations thereof.

Embodiment 26. The formula according to any one of Embodiments 24-25, wherein the formula comprises at least 1.1 mg/100 kcal, at least 1.2 mg/100 kcal, at least 1.3 mg/100 kcal, at least 1.4 mg/100 kcal, or at least 1.5 mg/100 kcal of the iron salt as elemental iron.

Embodiment 27. The formula according to any one of Embodiments 24-26, wherein the formula comprises no more than 1.6 mg/100 kcal, no more than 1.5 mg/100 kcal, no more than 1.4 mg/100 kcal, no more than 1.3 mg/100 kcal, or no more than 1.2 mg/100 kcal of the iron salt as elemental iron.

Embodiment 28. The formula according to any one of Embodiments 24-27, wherein the formula comprises the iron salt in a range of about 1.1-1.6 mg/100 kcal as elemental iron.

Embodiment 29. The formula according to any one of Embodiments 24-28, wherein the formula comprises the iron salt in a range of about 1.3-1.4 mg/100 kcal as elemental iron.

Embodiment 30. The formula according to any one of Embodiments 24-29, wherein the formula comprises L-methylfolate or its nutritionally acceptable salt and iron salt at a wt./100 kcal ratio in a range of from about 1:750 to about 1:3.

Embodiment 31. The formula according to any one of Embodiments 24-30, wherein the formula comprises L-methylfolate or its nutritionally acceptable salt and iron salt at a wt./100 kcal ratio in a range of from about 1:222 to about 1:20.

Embodiment 32. The formula according to any one of Embodiments 24-31, wherein the formula comprises iron salt and DHA at a wt./100 kcal ratio in a range of from about 1:333 to about 1:3.

Embodiment 33. The formula according to any preceding Embodiment, comprising an effective amount of methylcobalamin.

Embodiment 34. The formula according to Embodiment 33, wherein the formula comprises at least 0.23 μg/100 kcal, at least 0.25 μg/100 kcal, at least 0.3 μg/100 kcal, at least 0.35 μg/100 kcal, at least 0.4 μg/100 kcal, or at least 0.45 μg/100 kcal of methylcobalamin.

Embodiment 35. The formula according to any one of Embodiments 33-34, wherein the formula comprises no more than 0.5 μg/100 kcal, no more than 0.45 μg/100 kcal, no more than 0.4 μg/100 kcal, no more than 0.35 μg/100 kcal, no more than 0.3 μg/100 kcal, or no more than 0.25 μg/100 kcal of methylcobalamin.

Embodiment 36. The formula according to any one of Embodiments 33-35, wherein the formula comprises methylcobalamin in a range of about 0.23-0.5 μg/100 kcal.

Embodiment 37. The formula according to any one of Embodiments 33-36, wherein the formula comprises methylcobalamin in a range of about 0.25-0.35 μg/100 kcal.

Embodiment 38. The formula according to any preceding Embodiment, further comprising an effective amount of at least one of ascorbic acid, cholecalciferol, L-carnitine-L-tartrate, betaine, taurine, or nutritionally acceptable salts thereof, or combinations thereof.

Embodiment 39. The formula according to Embodiment 38, wherein the formula comprises at least 4 mg/100 kcal, at least 5 mg/100 kcal, at least 8 mg/100 kcal, at least 10 mg/100 kcal, at least 15 mg/100 kcal, at least 20 mg/100 kcal, or at least 25 mg/100 kcal of ascorbic acid or its nutritionally acceptable salt.

Embodiment 40. The formula according to any one of Embodiments 38-39, wherein the formula comprises no more than 30 mg/100 kcal, no more than 25 mg/100 kcal, no more than 20 mg/100 kcal, no more than 15 mg/100 kcal, no more than 10 mg/100 kcal, no more than 8 mg/100 kcal, or no more than 5 mg/100 kcal of ascorbic acid or its nutritionally acceptable salt.

Embodiment 41. The formula according to any one of Embodiments 38-40, wherein the formula comprises ascorbic acid or its nutritionally acceptable salt in a range of about 4-30 mg/100 kcal.

Embodiment 42. The formula according to any one of Embodiments 38-41, wherein the formula comprises ascorbic acid or its nutritionally acceptable salt in a range of about 8-30 mg/100 kcal.

Embodiment 43. The formula according to any one of Embodiments 38-42, wherein the formula comprises cholecalciferol in a range of about 1-3 μg/100 kcal.

Embodiment 44. The formula according to any one of Embodiments 38-43, wherein the formula comprises cholecalciferol in a range of about 2-2.5 μg/100 kcal.

Embodiment 45. The formula according to any one of Embodiments 38-44, wherein the formula comprises at least 1.2 mg/100 kcal, at least 1.5 mg/100 kcal, at least 2 mg/100 kcal, at least 2.5 mg/100 kcal, at least 3 mg/100 kcal, at least 3.5 mg/100 kcal, at least 4 mg/100 kcal, or at least 4.5 mg/100 kcal of L-carnitine-L-tartrate.

Embodiment 46. The formula according to any one of Embodiments 38-45, wherein the formula comprises no more than 5 mg/100 kcal, no more than 4.5 mg/100 kcal, no more than 4 mg/100 kcal, no more than 3.5 mg/100 kcal, no more than 3 mg/100 kcal, no more than 2.5 mg/100 kcal, no more than 2 mg/100 kcal, or no more than 1.5 mg/100 kcal of L-carnitine-L-tartrate.

Embodiment 47. The formula according to any one of Embodiments 38-46, wherein the formula comprises L-carnitine-L-tartrate in a range of about 1.2-5 mg/100 kcal.

Embodiment 48. The formula according to any one of Embodiments 38-47, wherein the formula comprises L-carnitine-L-tartrate in a range of about 1.5-3 mg/100 kcal.

Embodiment 49. The formula according to any one of Embodiments 38-48, wherein the formula comprises L-carnitine-L-tartrate and DHA at a wt./100 kcal ratio in a range of from about 1:42 to about 1:2.

Embodiment 50. The formula according to any one of Embodiments 38-49, wherein the formula comprises L-carnitine-L-tartrate and iron salt at a wt./100 kcal ratio in a range of from about 1:2.5 to about 33:1.

Embodiment 51. The formula according to any one of Embodiments 38-50, wherein the formula comprises L-carnitine-L-tartrate and betaine at a wt./100 kcal ratio in a range of from about 1:10 to about 5:1.

Embodiment 52. The formula according to any one of Embodiments 38-51, wherein the formula comprises L-carnitine-L-tartrate and taurine at a wt./100 kcal ratio in a range of from about 1:10 to about 5:1.

Embodiment 53. The formula according to any one of Embodiments 38-52, wherein the formula comprises L-carnitine-L-tartrate and L-methylfolate or its nutritionally acceptable salt at a wt./100 kcal ratio in a range of from about 1000:1 to about 25:1.

Embodiment 54. The formula according to any preceding Embodiment, further comprising at least one of soy lecithin, calcium phosphate, potassium citrate, sodium chloride, calcium carbonate, potassium hydroxide, potassium phosphate, magnesium chloride, potassium bicarbonate, ferrous sulfate, potassium chloride, zinc sulfate, cupric sulfate, manganese sulfate, potassium iodide, sodium selenite, choline bitartrate, ascorbyl palmitate, inositol, mixed tocopherol concentrate, dl-alpha tocopheryl acetate (vitamin E), niacinamide (vitamin B3), copper sulfate, mixed tocopherol concentrate, calcium pantothenate, vitamin A palmitate, riboflavin (vitamin B2), thiamine hydrochloride (vitamin B1), pyridoxine hydrochloride (vitamin B6), folic acid, phytonadione (vitamin K), biotin, cholecalciferol (vitamin D3), or combinations thereof.

Embodiment 55. The formula according to any preceding Embodiment, wherein the formula comprises L-5 methyl tetrahydrofolate (L-5-MTHF) constituting 100% by molar basis of the total folate label claim.

Embodiment 56. The formula according to any preceding Embodiment, wherein the formula comprises L-5-MTHF constituting 90% by molar basis of the total folate label claim, and folic acid constituting 10% by molar basis of the total folate label claim.

Embodiment 57. The formula according to any preceding Embodiment, wherein the formula comprises L-5-MTHF constituting 80% by molar basis of the total folate label claim, and folic acid constituting 20% by molar basis of the total folate label claim.

Embodiment 58. The formula according to any preceding Embodiment, wherein the formula comprises L-5-MTHF constituting 70% by molar basis of the total folate label claim, and folic acid constituting 30% by molar basis of the total folate label claim.

Embodiment 59. The formula according to any preceding Embodiment, wherein the formula comprises L-5-MTHF constituting 60% by molar basis of the total folate label claim, and folic acid constituting 40% by molar basis of the total folate label claim.

Embodiment 60. The formula according to any preceding Embodiment, wherein the formula comprises L-5-MTHF constituting 50% by molar basis of the total folate label claim, and folic acid constituting 50% by molar basis of the total folate label claim.

Embodiment 61. The formula according to any preceding Embodiment, wherein the formula comprises L-5-MTHF constituting 40% by molar basis of the total folate label claim, and folic acid constituting 60% by molar basis of the total folate label claim.

Embodiment 62. The formula according to any preceding Embodiment, wherein the formula comprises L-5-MTHF constituting 30% by molar basis of the total folate label claim, and folic acid constituting 70% by molar basis of the total folate label claim.

Embodiment 63. The formula according to any preceding Embodiment, wherein the formula comprises L-5-MTHF constituting 20% by molar basis of the total folate label claim, and folic acid constituting 80% by molar basis of the total folate label claim.

Embodiment 64. The formula according to any preceding Embodiment, wherein the formula comprises L-5-MTHF constituting 10% by molar basis of the total folate label claim, and folic acid constituting 90% by molar basis of the total folate label claim.

Embodiment 65. The formula according to any preceding Embodiment, wherein the formula comprises ferric pyrophosphate constituting 100% by molar basis of the total elemental iron label claim.

Embodiment 66. The formula according to any preceding Embodiment, wherein the formula comprises ferric pyrophosphate constituting 90% by molar basis of the total elemental iron label claim, and another iron form constituting 10% by molar basis of the total elemental iron label claim.

Embodiment 67. The formula according to any preceding Embodiment, wherein the formula comprises ferric pyrophosphate constituting 80% by molar basis of the total elemental iron label claim, and another iron form constituting 20% by molar basis of the total elemental iron label claim.

Embodiment 68. The formula according to any preceding Embodiment, wherein the formula comprises ferric pyrophosphate constituting 70% by molar basis of the total elemental iron label claim, and another iron form constituting 30% by molar basis of the total elemental iron label claim.

Embodiment 69. The formula according to any preceding Embodiment, wherein the formula comprises ferric pyrophosphate constituting 60% by molar basis of the total elemental iron label claim, and another iron form constituting 40% by molar basis of the total elemental iron label claim.

Embodiment 70. The formula according to any preceding Embodiment, wherein the formula comprises ferric pyrophosphate constituting 50% by molar basis of the total elemental iron label claim, and another iron form constituting 50% by molar basis of the total elemental iron label claim.

Embodiment 71. The formula according to any preceding Embodiment, wherein the formula comprises ferric pyrophosphate constituting 40% by molar basis of the total elemental iron label claim, and another iron form constituting 60% by molar basis of the total elemental iron label claim.

Embodiment 72. The formula according to any preceding Embodiment, wherein the formula comprises ferric pyrophosphate constituting 30% by molar basis of the total elemental iron label claim, and another iron form constituting 70% by molar basis of the total elemental iron label claim.

Embodiment 73. The formula according to any preceding Embodiment, wherein the formula comprises ferric pyrophosphate constituting 20% by molar basis of the total elemental iron label claim, and another iron form constituting 80% by molar basis of the total elemental iron label claim.

Embodiment 74. The formula according to any preceding Embodiment, wherein the formula comprises ferric pyrophosphate constituting 10% by molar basis of the total elemental iron label claim, and another iron form constituting 90% by molar basis of the total elemental iron label claim.

Embodiment 75. The formula according to any one of Embodiments 66-74, wherein the another iron form is selected from ferrous sulfate, ferrous bisglycinate chelate, or combinations thereof.

Embodiment 76. The formula according to any preceding Embodiment, wherein the formula comprises ferric pyrophosphate constituting 100% by molar basis of the total elemental iron label claim, and L-5-MTHF constituting 100% by molar basis of the total folate label claim.

Embodiment 77. The formula according to any preceding Embodiment, wherein the formula comprises ferric pyrophosphate constituting 100% by molar basis of the total elemental iron label claim, and L-5-MTHF constituting 50% by molar basis of the total folate label claim and folic acid constituting 50% by molar basis of the total folate label claim.

Embodiment 78. The formula according to any preceding Embodiment, wherein the formula comprises ferric pyrophosphate constituting 50% by molar basis of the total elemental iron label claim and ferrous sulfate constituting 50% by molar basis of the total elemental iron label claim, and L-5-MTHF constituting 100% by molar basis of the total folate label claim.

Embodiment 79. The formula according to any preceding Embodiment, wherein the formula comprises ferric pyrophosphate constituting 50% by molar basis of the total elemental iron label claim and ferrous sulfate constituting 50% by molar basis of the total elemental iron label claim, and L-5-MTHF constituting 50% by molar basis of the total folate label claim and folic acid constituting 50% by molar basis of the total folate label claim.

Embodiment 80. The formula according to any preceding Embodiment, wherein the formula can be in powdered form or in liquid form.

Embodiment 81. A method for promoting brain, eye and/or immune system development in an infant or a toddler, comprising administering a nutrient formula according to any preceding Embodiment to the infant or toddler.

Embodiment 82. The method according to Embodiment 81, comprising administering to an infant that is preterm.

### DOCUMENTS CITED

1. Abbaspour N, Hurrell R, Kelishadi R. Review on iron and its importance for human health. J Res Med Sci. 2014; 19 (2): 164-174.
2. Aisen P, Enns C, Wessling-Resnick M. Chemistry and biology of eukaryotic iron metabolism. Int J Biochem Cell Biol. 2001 October; 33 (10): 940-59. doi: 10.1016/s1357-2725 (01) 00063-2. PMID: 11470229.
3. Baker, R D and Greer, F. The Committee on Nutrition; Diagnosis and Prevention of Iron Deficiency and Iron-Deficiency Anemia in Infants and Young Children (0-3 Years of Age). Pediatrics November 2010; 126 (5): 1040-1050. 10.1542/peds.2010-2576
4. Berry R J, Li Z, Erickson J D, Li S, Moore C A, Wang H, Mulinare J, Zhao P, Wong L Y, Gindler J, Hong S X, Correa A. Prevention of neural-tube defects with folic acid in China. China-U.S. Collaborative Project for Neural Tube Defect Prevention. N Engl J Med. 1999 Nov. 11; 341 (20): 1485-90. doi: 10.1056/NEJM199911113412001. Erratum in: N Engl J Med. 1999 Dec. 9; 341 (24): 1864. PMID: 10559448.
5. Birch E E, Garfield S, Hoffman D R, Uauy R, Birch D G. A randomized controlled trial of early dietary supply of long-chain polyunsaturated fatty acids and mental development in term infants. Dev Med Child Neurol. 2000 March; 42 (3): 174-81. doi: 10.1017/s0012162200000311. PMID: 10755457
6. Blencowe H, Cousens S, Modell B, Lawn J. Folic acid to reduce neonatal mortality from neural tube disorders. Int J Epidemiol. 2010 April; 39 Suppl 1 (Suppl 1): i110-21. doi: 10.1093/ije/dyq028. PMID: 20348114; PMCID: PMC2845867.
7. Bradbury J. Docosahexaenoic acid (DHA): an ancient nutrient for the modern human brain. Nutrients. 2011 May; 3 (5): 529-54. doi: 10.3390/nu3050529. Epub 2011 May 10. PMID: 22254110; PMCID: PMC3257695.
8. Briguglio M, Hrelia S, Malaguti M, et al. The Central Role of Iron in Human Nutrition: From Folk to Contemporary Medicine. Nutrients. 2020; 12 (6): 1761. Published 2020 Jun. 12. doi: 10.3390/nu12061761
9. Büttner B E, Witthöft C M, Domellof M, Hernell O, Öhlund I. Effect of type of heat treatment of breastmilk on folate content and pattern. Breastfeed Med. 2014 March; 9 (2): 86-91. doi: 10.1089/bfm.2013.0008. Epub 2013 Jun. 20. PMID: 23786311.
10. Cui R, Iso H, Date C, Kikuchi S, Tamakoshi A; Japan Collaborative Cohort Study Group. Dietary folate and vitamin b6 and B12 intake in relation to mortality from cardiovascular diseases: Japan collaborative cohort study. Stroke. 2010 June; 41 (6): 1285-9. doi: 10.1161/STROKEAHA. 110.578906. Epub 2010 Apr. 15. PMID: 20395608.
11. Dallman P R. Iron deficiency anemia: a synthesis of current scientific knowledge and U.S. recommendations for prevention and treatment. In: Earl R, Woteki C E, eds. Iron Deficiency Anemia: Recommended Guidelines for Prevention, Detection and Management Among U.S. Children and Women of Childbearing Age. Washington, DC: National Academies Press; 1993:41-97.
12. De Wals P, Tairou F, Van Allen M I, Lowry R B, Evans J A, Van den Hof M C, Crowley M, Uh S H, Zimmer P, Sibbald B, Fernandez B, Lee N S, Niyonsenga T. Spina bifida before and after folic acid fortification in Canada. Birth Defects Res A Clin Mol Teratol. 2008 September; 82 (9): 622-6. doi: 10.1002/bdra.20485. PMID: 18655127.
13. Eussen S, Alles M, Uijterschout L, Brus F, van der Horst-Graat J. Iron intake and status of children aged 6-36 months in Europe: a systematic review. Ann Nutr Metab. 2015; 66 (2-3): 80-92. doi: 10.1159/000371357. Epub 2015 Jan. 21. PMID: 25612840.
14. Friel J K, Aziz K, Andrews W L, Harding S V, Courage M L, Adams R J. A double-masked, randomized control trial of iron supplementation in early infancy in healthy term breast-fed infants. J Pediatr. 2003 November; 143 (5): 582-6. doi: 10.1067/S0022-3476 (03) 00301-9. PMID: 14615726.
15. Georgieff M K, Wewerka S W, Nelson C A, Deregnier R A. Iron status at 9 months of infants with low iron stores at birth. J Pediatr. 2002 September; 141 (3): 405-9. doi: 10.1067/mpd.2002.127090. PMID: 12219063.
16. Hadley K B, Ryan A S, Forsyth S, Gautier S, Salem N Jr. The Essentiality of Arachidonic Acid in Infant Development. Nutrients. 2016; 8 (4): 216. Published 2016 Apr. 12. doi: 10.3390/nu8040216.
17. Horrocks L A, Yeo Y K. Health benefits of docosahexaenoic acid (DHA). Pharmacol Res. 1999 September; 40 (3): 211-25. doi: 10.1006/phrs. 1999.0495. PMID: 10479465.
18. Innis S M. Essential fatty acids in growth and development. Prog Lipid Res. 1991; 30 (1): 39-103. doi: 10.1016/0163-7827 (91) 90006-q. PMID: 1771170.
19. Lien E L, Richard C, Hoffman D R. DHA and ARA addition to infant formula: Current status and future research directions. Prostaglandins Leukot Essent Fatty Acids. 2018 January; 128:26-40. doi: 10.1016/j.plefa.2017.09.005. Epub 2017 Sep. 15. PMID: 29413359.
20. Lieu P T, Heiskala M, Peterson P A, Yang Y. The roles of iron in health and disease. Mol Aspects Med. 2001 February-April; 22 (1-2): 1-87. doi: 10.1016/s0098-2997 (00) 00006-6. PMID: 11207374.
21. Linda Schaeffer, Henning Gohlke, Martina Müller, Iris M. Heid, Lyle J. Palmer, Iris Kompauer, Hans Demmelmair, Thomas Illig, Berthold Koletzko, Joachim Heinrich, Common genetic variants of the FADS1 FADS2 gene cluster and their reconstructed haplotypes are associated with the fatty acid composition in phospholipids, Human Molecular Genetics, Volume 15, Issue 11, 1 Jun. 2006, Pages 1745-1756, https://doi.org/10.1093/hmq/ddl117.
22. Meinzen-Derr J K, Guerrero M L, Altaye M, Ortega-Gallegos H, Ruiz-Palacios G M, Morrow A L. Risk of infant anemia is associated with exclusive breast-feeding and maternal anemia in a Mexican cohort. J Nutr. 2006 February; 136 (2): 452-8. doi: 10.1093/jn/136.2.452. PMID: 16424127.
23. Naderi N, House J D. Recent Developments in Folate Nutrition. Adv Food Nutr Res. 2018; 83:195-213. doi: 10.1016/bs.afnr.2017.12.006. Epub 2018 Feb. 2. PMID: 29477222.
24. Rosenblatt D S. Inherited disorders of folate transport and metabolism. In: Seriver A L, Beaudet W S, Sly W S, Valle D, editors. The metabolic basis of inherited disease. 2. New York: McGraw-Hill; 1995. p. 3111-28.
25. Schlotz W, Jones A, Phillips D I, Gale C R, Robinson S M, Godfrey K M. Lower maternal folate status in early pregnancy is associated with childhood hyperactivity and peer problems in offspring. J Child Psychol Psychiatry. 2010 May; 51 (5): 594-602. doi: 10.1111/j. 1469-7610.2009.02182.x. Epub 2009 Oct. 28. PMID: 19874428; PMCID: PMC2862762.
26. Scott D T, Janowsky J S, Carroll R E, Taylor J A, Auestad N, Montalto M B. Formula supplementation with long-chain polyunsaturated fatty acids: are there developmental benefits? Pediatrics. 1998 November; 102 (5): E59. doi: 10.1542/peds. 102.5.e59. PMID: 9794989.
27. Serrano-Amatriain C, Ledesma-Amaro R, López-Nicolás R, Ros G, Jiménez A, Revuelta J L. Folic Acid Production by Engineered Ashbya gossypii. Metab Eng. 2016 November; 38:473-482. doi: 10.1016/j.ymben.2016.10.011. Epub 2016 Oct. 28. PMID: 27989803.
28. Shane B. Folate and vitamin B12 metabolism: overview and interaction with riboflavin, vitamin B6, and polymorphisms. Food and Nutrition Bulletin. 2008; 29 (2 Suppl): S5-16. Epub 2008 Aug. 20. PubMed https://doi.org/10.1177/15648265080292S103 PMID: 18709878.
29. Sherry B, Mei Z, Yip R. Continuation of the decline in prevalence of anemia in low-income infants and children in five states. Pediatrics. 2001 April; 107 (4): 677-82. doi: 10.1542/peds. 107.4.677. PMID: 11335743
30. Smith A D. The worldwide challenge of the dementias: a role for B vitamins and homocysteine? Food Nutr Bull. 2008 June; 29 (2 Suppl):S143-72. doi: 10.1177/15648265080292S119. PMID: 18709889.
31. Tai E K, Wang X B, Chen Z Y. An update on adding docosahexaenoic acid (DHA) and arachidonic acid (AA) to baby formula. Food Funct. 2013 December; 4 (12): 1767-75. doi: 10.1039/c3fo60298b. Epub 2013 Oct. 22. PMID: 24150114.
32. Tounian P, Bellaiche M, Legrand P. ARA or no ARA in infant formulae, that is the question. Arch Pediatr. 2021 January; 28 (1): 69-74. doi: 10.1016/j.arcped.2020.10.001. Epub 2020 Oct. 22. PMID: 33268182.
33. Troesch B, Demmelmair J, Gimpfl M, Hecht C, Lakovic G, Roehle R, Sipka L, Trisic B, Vusurovic M, Schoop R, Zdjelar S, Koletzko B; MEFOLIN Study Group. Suitability and safety of L-5-methyltetrahydrofolate as a folate source in infant formula: A randomized-controlled trial. PLOS One. 2019 Aug. 19; 14 (8):e0216790. doi: 10.1371/journal.pone.0216790. PMID: 31425504; PMCID: PMC6699731.
34. Uauy, R., Mena, P., Wegher, B. et al. Long Chain Polyunsaturated Fatty Acid Formation in Neonates: Effect of Gestational Age and Intrauterine Growth. Pediatr Res 47, 127 (2000). https://doi.org/10.1203/00006450-200001000-00022
35. Weisberg I, Tran P, Christensen B, Sibani S, Rozen R. A second genetic polymorphism in methylenetetrahydrofolate reductase (MTHFR) associated with decreased enzyme activity. Mol Genet Metab. 1998; 64 (3): 169-72. https://doi.org/10.1006/mgme.1998.2714 PMID: 9719624.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A nutritional formula for an infant or a toddler, comprising an effective amount of L-methylfolate or its nutritionally acceptable salt.

2. The formula according to claim 1, wherein the nutritionally acceptable salt of L-methylfolate is selected from the group consisting of calcium L-methylfolate, monosodium L-methylfolate, glucosamine L-methylfolate, magnesium L-methylfolate, or combinations thereof.

3. The formula according to claim 1, wherein the nutritionally acceptable salt of L-methylfolate is calcium L-methylfolate.

4. The formula according to any one of claims 1-3, wherein the formula comprises the L-methylfolate or its nutritionally acceptable salt in a range of about 15.5-48 μg/100 kcal.

5. The formula according to any one of claims 1-4, comprising an effective amount of docosahexaenoic acid (DHA).

6. The formula according to claim 5, wherein the formula comprises Schizochytrium oil as a source of DHA.

7. The formula according to any one of claims 5-6, wherein the formula comprises DHA in a range of about 23-40 mg/100 kcal.

8. The formula according to any one of claims 1-7, comprising an effective amount of arachidonic acid (ARA).

9. The formula according to claim 8, wherein the formula comprises Mortierella alpina oil as a source of ARA.

10. The formula according to any one of claims 8-9, wherein the formula comprises ARA in a range of about 35-80 mg/100 kcal.

11. The formula according to any one of claims 5-10, wherein the formula comprises DHA and ARA at a wt./100 kcal ratio in a range of from about 1:1 to about 1:2.

12. The formula according to any one of claims 1-11, comprising an effective amount of iron salt as a source of elemental iron.

13. The formula according to claim 12, wherein the iron salt is selected from the group consisting of iron (III) pyrophosphate (ferric pyrophosphate), iron (II) fumarate (ferrous fumarate), iron (II) gluconate (ferrous gluconate), iron (II) sulfate (ferrous sulfate), iron (II) bisglycinate (ferrous bisglycinate chelate), or combinations thereof.

14. The formula according to any one of claims 12-13, wherein the formula comprises the iron salt in a range of about 1.3-1.4 mg/100 kcal as elemental iron.

15. The formula according to any one of claims 1-14, comprising an effective amount of methylcobalamin.

16. The formula according to claim 15, wherein the formula comprises methylcobalamin in a range of about 0.25-0.35 μg/100 kcal.

17. The formula according to any one of claims 1-16, further comprising an effective amount of at least one of ascorbic acid, cholecalciferol, L-carnitine-L-tartrate, betaine, taurine, or nutritionally acceptable salts thereof, or combinations thereof.

18. The formula according to any one of claims 1-17, further comprising at least one of soy lecithin, calcium phosphate, potassium citrate, sodium chloride, calcium carbonate, potassium hydroxide, potassium phosphate, magnesium chloride, potassium bicarbonate, ferrous sulfate, potassium chloride, zinc sulfate, cupric sulfate, manganese sulfate, potassium iodide, sodium selenite, choline bitartrate, ascorbyl palmitate, inositol, mixed tocopherol concentrate, dl-alpha tocopheryl acetate (vitamin E), niacinamide (vitamin B3), copper sulfate, mixed tocopherol concentrate, calcium pantothenate, vitamin A palmitate, riboflavin (vitamin B2), thiamine hydrochloride (vitamin B1), pyridoxine hydrochloride (vitamin B6), folic acid, phytonadione (vitamin K), biotin, cholecalciferol (vitamin D3), or combinations thereof.

19. The formula according to any one of claims 1-18, wherein the formula comprises L-5 methyl tetrahydrofolate (L-5-MTHF) constituting 100% by molar basis of the total folate label claim.

20. The formula according to any one of claims 1-18, wherein the formula comprises L-5-MTHF constituting 50% by molar basis of the total folate label claim, and folic acid constituting 50% by molar basis of the total folate label claim.

21. The formula according to any one of claims 1-20, wherein the formula comprises ferric pyrophosphate constituting 100% by molar basis of the total elemental iron label claim.

22. The formula according to any one of claims 1-20, wherein the formula comprises ferric pyrophosphate constituting 50% by molar basis of the total elemental iron label claim, and another iron form constituting 50% by molar basis of the total elemental iron label claim.

23. A nutritional formula for an infant or a toddler, comprising ferric pyrophosphate constituting 100% by molar basis of the total elemental iron label claim, and L-5-MTHF constituting 100% by molar basis of the total folate label claim.

24. A nutritional formula for an infant or a toddler, comprising ferric pyrophosphate constituting 100% by molar basis of the total elemental iron label claim, and L-5-MTHF constituting 50% by molar basis of the total folate label claim and folic acid constituting 50% by molar basis of the total folate label claim.

25. A nutritional formula for an infant or a toddler, comprising ferric pyrophosphate constituting 50% by molar basis of the total elemental iron label claim and ferrous sulfate constituting 50% by molar basis of the total elemental iron label claim, and L-5-MTHF constituting 100% by molar basis of the total folate label claim.

26. A nutritional formula for an infant or a toddler, comprising ferric pyrophosphate constituting 50% by molar basis of the total elemental iron label claim and ferrous sulfate constituting 50% by molar basis of the total elemental iron label claim, and L-5-MTHF constituting 50% by molar basis of the total folate label claim and folic acid constituting 50% by molar basis of the total folate label claim.

27. The formula according to any one of claims 1-26, wherein the formula can be in powdered form or in liquid form.

28. The formula according to any one of claims 1-27, wherein the formula is for a preterm or full term infant.

29. A method for promoting brain, eye and/or immune system development in an infant or a toddler, comprising administering a nutrient formula according to any preceding Embodiment to the infant or toddler.

30. The method according to claim 29, comprising administering to an infant that is preterm.
